(19) United States
(12) Patent Application Publication
(10) Pub. No.: US 2007/0081947 A1
(43) Pub. Date: Apr. 12, 2007

(54) Solid active ingredient formulation

(75) Inventors: Axel Eble, Koln (DE); Peter Baur, Schondorf (DE); Karl Reizlein, Monheim (DE)

Correspondence Address: NORRIS, MCLAUGHLIN & MARCUS, P.A., 875 THIRD AVE, 18TH FLOOR, NEW YORK, NY, 10022, US

(73) Assignee: Bayer Technology Service GmbH, Leverkusen (DE)

(21) Appl. No.: 10/577,608

(22) PCT Filed: Oct. 19, 2004
(86) PCT No.: PCT/EP04/11807

§ 371 Date: Nov. 30, 2006

(30) Foreign Application Priority Data
Oct. 31, 2003 (DE) 103 51 087.7

(51) Int. Cl. A01N 25/00; A61K 9/12

(52) U.S. Cl. 424/45; 424/405

(57) ABSTRACT

The present invention relates to novel solid active substance formulations comprising solid active substances, dispersants, and polymers which together result in a fine-particle, predominantly amorphous mixture, to a process for the preparation thereof and to the use thereof for application of the contained bioactive active substances.

The present invention relates to novel solid active substance formulations comprising solid active substances, dispersants, and polymers which together result in a fine-particle, predominantly amorphous mixture, to a process for the preparation thereof and to the use thereof for application of the contained bioactive active substances.

The efficacy of slightly soluble active substances is greatly restricted by the greatly restricted transport from the application site to the site of the desired effect in the biological system. Numerous approaches to improving the solubility or generally the bioavailability of such slightly soluble active substances by suitable measures in the formulation has been disclosed.

Thus, Müller et al., Pharm. Ind. 61, No. 1:74-78 (1999) describe how the rate of dissolution can be improved by grinding crystalline active substances in high-pressure homogenizers to give so-called nanosuspensions through enlarging the surface area, through increased saturation solubility, and through shortening the diffusion distance. G. G. Liversidge et al., Int. J. Pharm. 125: 91 (1995) describe in a similar manner the possibility of improving the rate of dissolution of crystalline active substances by grinding in ball mills to give fine-particle suspensions.

A further improvement in the bioavailability of slightly soluble active substances is reported in H. Auweter et al., Angew. Chem. Int. Ed. 38, No. 15: 2188-91 (1999), by preparing the active substance in fine-particle and additionally X-ray amorphous form by precipitation, and stabilizing this state by a shell enveloping the fine-particle amorphous active substance particles. The solubility of a substance in the amorphous state is greater than the solubility in the crystalline state. In addition to the advantages described above of fine-particle formulations, therefore, formulation in the amorphous state represents a further advantage. EP 0065193A2 and EP 0932339B1 also relate to fine-particle amorphous core-shell particles defined above.

It has now emerged that the fine-particle amorphous state cannot be stabilized equally easily and sufficiently long-term by a corresponding shell layer for all active substances. It was an object of the present development to stabilize the fine-particle amorphous state of the active substances in another way in order thus to link the advantages, defined above, of better bioavailability with the advantages of better storage stability wider applicability to different active substances.

Novel active substance formulations in powder form have now been found and consist of
at least one active substance which is solid at room temperature,
at least one dispersant,
at least one polymer and
where appropriate additives,
   are in the amorphous state and have diameters in the nanometer range. Active substance, dispersant and polymer form therein the predominantly amorphous mixed phase. To improve handling, these particles may additionally be embedded in a carrier.

“Predominantly amorphous” means that more than half, preferably more than 70%, of the active substance is in amorphous form in the formulation of the invention. It is possible to determine, as measure of the amorphous state, conversely the degree of crystallinity in a simple manner known to the skilled worker by differential thermal analysis (or differential scanning calorimetry, DSC).

The invention relates to a process for preparing amorphous mixtures based on crystalline active substances, in particular active substance formulations based on crystalline active substances, with the steps of
a) complete dissolution of the active substance A) in a solvent 1, where appropriate together with a dispersing aid C) to form a solution E).
b) provision of a displacement agent 2, in particular a liquid 2, in which the solubility of the active substance A) is less than 1% by weight and which is miscible with solvent 1 and which effects precipitation of the active substance A), as solution F.
c) addition of a polymer B), in particular predominantly amorphous polymers which are readily soluble in water, particularly preferably selected from the series: dextrans, dextrins, gum Arabic, polyvinyl alcohol, polyvinylpyrrolidone, polyethylene glycol, polyaspartic acid and alginates, to the solution from step a) and/or to solution F) from step b).
d) mixing of two solvent streams of solutions E) and F), preferably in a mixing nozzle, with the two part-streams being fed continuously and uniformly to the mixing zone, where appropriate to form turbulent flow in the region of the mixing zone.
e) removal of the solvents from the mixture by, in particular, freeze drying, spray drying or spray granulation.

The mixing in step d) and, where appropriate, formation of a turbulent flow is preferably effected by a pressure gradient across the mixing nozzle, by stirring or by ultrasound treatment of the mixed streams.

The viscosity of solutions E) and F) is kept in particular below 100 mPas.

The displacement agent 2 may be in particular water or an aqueous solution of an acid, of a base or of a salt.

The solvent 1 can preferably be a low molecular weight organic solvent, in particular one selected from the series of short-chain alcohols having 1 to 10 carbon atoms, such as, for example, methanol, ethanol, 2-propanol, of short-chain glycols, such as, for example, ethylene glycol, 1,2-propylene glycol, of short-chain ketones having 3 to 10 carbon atoms, such as, for example, acetone, 2-butanone, carboxylic acids such as, for example, acetic acid, ethers such as, for example, diethyl ether, tetrahydrofuran or methyl tert-butyl ether, esters such as, for example, methyl acetate, ethyl actetate or methyl formate, heterocyclic amines such as, for example, pyridines, formamides such as, for example, dimethylformamide, or else n-methylpyrrolidone or dimethyl sulfoxide or an aqueous solution of a base or of an acid. The aforementioned solvents may in each case be used alone or in a mixture.

In a preferred variant of the process, the drying step e) is preceded by addition of from 10 to 30% by weight of a carrier selected from the series talc, polyethylene glycol, modified starch or high molecular weight sugar, where appropriate also further polymer B), in each case based on the total weight of the formulation, to the suspension.

Active substance A) can be any active substance which is slightly soluble in pure water, and its proportion in the finished formulation is 0.5-50% by weight, preferably 5-30% by weight, based on the mixture.

Dispersant C) or mixtures of dispersants are specifically selected for the active substance. The proportion of the total amount of all dispersants C) in relation to the amount of active substance A) is from 0.1 times to 5 times, preferably 0.25 times to 3 times, particularly preferably 0.5 times to, 2 times.

The proportion of the total of polymers B) in the finished formulation is 5-90% by weight, preferably 10-80% by weight, particularly preferably 15-75% by weight.

Possible further additives are customary additives and auxiliaries known in principle for formulations, such as plasticizers, swelling agents or preservatives.

Suitable dispersants C) can be found in a simple manner known to the skilled worker by, for example, observing the sedimentation behavior. For this purpose, crystalline or amorphous active substance is ground and suspended in equal portions with a selection of dispersants in water (e.g. in each case 0.2 g of active substance with 0.2 g of dispersant in 15 ml of water). The suspension is then redispersed by treatment with ultrasound, and the effect of the dispersant is observed by means of the sedimentation behavior. Suitable dispersants C) are distinguished by greatly delaying or suppressing the sedimentation of the particulate active substance A). A suitable dispersant is for example one which prevents sedimentation for up to 30 min. Preliminary restriction of the choice of dispersants C) to be tested is possible by examining the electrochemical interface potential of the active substance A) in an aqueous environment and by examining the interactions to be expected between the dispersant and the active substance molecule.

Suitable dispersants C) for the mixtures of the invention are all conventional nonionic, anionic, cationic and zwitterionic substances having the surface-active properties which are normally employed in formulations. These substances include products of the reaction of fatty acids, fatty acid esters, fatty alcohols, fatty amines, alkylphenols or alkylarylphenols with ethylene oxide and/or propylene oxide, and the sulfuric esters, phosphoric monoesters and phosphoric diesters thereof, and additionally alkyl sulfonates, alkyl sulfates, aryl sulfates, alkylaryl sulfates, alkylether sulfates, alkylarylether sulfates, tetraalkylammonium halides, trialkylarylammonium halides, alkylaryl ethoxylates, sorbitan ethoxylates and alkylamine sulfonates. The dispersants C) can be employed singly or else in a mixture. Mention may preferably also be made of products of the reaction of castor oil with ethylene oxide in the molar ratio 1:20 to 1:60, products of the reaction of C₆-C₂₀-alcohols with ethylene oxide in the molar ratio 1:5 to 1:50, products of the reaction of fatty amines with ethylene oxide in the molar ratio 1:2 to 1:20, products of the reaction of 1 mol of phenol with 2 to 3 mol of styrene and 10 to 50 mol of ethylene oxide, products of the reaction of C₈-C₁₂-alkylphenols with ethylene oxide in the molar ratio 1:5 to 1:30, alkyl glycosides, C₈-C₁₆-alkylbenzene sulfonic acid salts such as, for example, calcium, monoethanolammonium, diethanolammonium and triethanolammonium salts.

Examples which may be mentioned of nonionic dispersants C) are the products known under the names Pluronic PE 10 100 and Pluronic F 68 (from BASF) and Atlox 4913 (from Uniqema). Also suitable are tristyrylphenyl ethoxylates. Examples which may be mentioned of anionic dispersants C) are the Bayer AG product commercially available under the name Baykanol SL (=product of the condensation of sulfonated ditolyl ether with formaldehyde), and phosphated or sulfated tristyrylphenol ethoxylates, with specific mention for Soprophor SLK and Soprophor 4D 384 (from Rhodia).

Examples of dispersants C) which may additionally be mentioned are copolymers of ethylene oxide and propylene oxide, products of the reaction of tristyrylphenol with ethylene oxide and/or propylene oxide, such as tristyrylphenol ethoxylate having an average of 24 ethylene oxide groups, tristyrylphenol ethoxylate having an average of 54 ethylene oxide groups or tristyrylphenol ethoxylate propoxylate having an average of 6 ethylene oxide and 8 propylene oxide groups, in addition phosphated or sulfated tristyrylphenol ethoxylates such as phosphated tristyrylphenol ethoxylate having an average of 16 ethylene oxide groups, sulfated tristyrylphenol ethoxylate having an average of 16 ethylene oxide groups or ammonium salt of phosphated tristyrylphenol ethoxylate having an average of 16 ethylene oxide groups, also lipoids such as phospholipid sodium glycolate or lecithin, and also liguinsulfonates. Also suitable in addition are substances having wetting properties. Mention may preferably be made of alkylphenol ethoxylates, dialkyl sulfosuccinates, such as diisooctyl sulfosuccinate sodium, lauryl ether sulfates and polyoxyethylene sorbitan fatty acid esters.

Suitable polymers B) for use in formulations of the invention are predominantly amorphous polymers which are readily soluble in water, especially highly polar polymers, in particular those having different polar functional groups. Those which may be mentioned are in particular dextrans, dextrins, gum Arabic, polyvinyl alcohol, polyvinylpyrrolidone, polyethylene glycol, polyaspartic acid and alginates. Suitable in this connection are both single ones of these polymers B) and any mixtures of said polymers B).

The term “polyvinyl alcohol” means in the present case both water-soluble vinyl alcohol polymerization products and water-soluble, partially hydrolyzed polymers of vinyl acetate, preferably having a proportion of acetate groups of between 1 and 28%, particularly preferably having a proportion of acetate groups of between 15 and 28%. Polyvinyl alcohol having an average molecular weight of between 10 000 and 200 000 is preferred, particularly preferably between 13 000 and 130 000.

The term “polyvinylpyrrolidone” means in the present case vinylpyrrolidone/vinyl acetate copolymers having a between 10 000 and 200 000, preferably between 24 000 and 55 000.

Polymers suitable for a specific active substance/dispersant pairing can be found in the manner known to the skilled worker on the basis of the criterion of maximal miscibility. The miscibility can be assessed for example by use of the glass transition points ascertained by differential thermal analysis. If separate amorphous phases are present, these are generally distinguished by separate glass transition points. If, by contrast, a mixed phase forms, this can be identified for example with a glass transition point which lies between the glass transition points of the respective starting materials.

The invention further relates to an amorphous mixture based on crystalline active substances, in particular active substance formulation, consisting at least of
0.5 to 50% by weight, in particular 5 to 30% by weight, of an active substance A) which is usually crystalline at 50° C.,
50 to 90% by weight, preferably 10 to 80% by weight, particularly preferably 15 to 75% by weight, of a polymer B), in particular selected from the series: dextrans, dextrins, gum Arabic, polyvinyl alcohol, polyvinylpyrrolidone, polyethylene glycol, polyaspartic acid and alginates
and, based on the proportion of active substance A), 0.1 to 5 times, preferably 0.25 to 3 times, particularly preferably 0.5 to 2 times, of a dispersing aid C), in particular of a nonionic, anionic, cationic or zwitterionic surface-active compound,
characterized in that the mixture comprises homogeneous primary particles of a mixture of the substances A), B), C) having an average particle diameter of <5 μm, preferably <2 μm, particularly preferably <1 μm, where more than 50% of the active substance A) therein is present in the amorphous state.

The dispersing aid C) is preferably selected from the series: products of the reaction of fatty acids, fatty acid esters, fatty alcohols, fatty amines, alkylphenols or alkylarylphenols with ethylene oxide and/or propylene oxide, and their sulfuric esters, phosphoric acid, monoesters and phosphoric diesters, products of the reaction of ethylene oxide with propylene oxide alkylsulfonates, alkyl sulfates, aryl sulfates, alkylaryl sulfates, alkyl ether sulfates, alkylaryl ether sulfates, tetraalkylammonium halides, trialkylarylammonium halides, alkylaryl ethoxylate, sorbitan ethoxylates and alkylamine sulfonates alone or in any mixture.

Preference is given to an active substance-containing mixture characterized in that the active substance is selected from the series of crop protection agents such as, for example, herbicides, fungicides, insecticides, acaricides, nematicides, bird repellents, plant nutrients and soil conditioners. Examples which may be mentioned in this connection are bistrifluron, boramsulfuron, mesosulfuronmethyl, pyraclostrobin, pyriftalid, abamectin, AC 94,377, acequinocyl, acibenzolar-S-methyl, aclonifen, acrinathrin, AKH-7088, amidosulfuron, amitraz, anilofos, anthraquinone, atrazine, azafenidin, azinphosmethyl, azocyclotin, azoxystrobin, beflubutamid, benalaxyl, benazolinethyl, benfluralin, benomyl, benoxacor, bensulfuron-methyl, bensultap, benzobicyclon, benzofenap, benzoximate, bifenazate, bifenox, bifenthrin, bitertanol, brodifacoum, bromadiolone, bromethalin, bromobutide, bromopropylate, bromuconazole, bupirimate, buprofezin, butafenacil, butralin, butroxydim, cafenstrole, captafol, captan, carbendazim, carpropamid, chinomethionat, chlorbromuron, chlordane, chlorfluazuron, chlorflurenol-methyl, chlorimuron-ethyl, chlorothalonil, chlorthal-dimethyl, chlozolinate, chromafenozide, cinidon-ethyl, clodinafop-propargyl, clofentezine, clomeprop, cloquintocet-mexyl, cloransulam-methyl, copper oxychloride, copper sulfate (tribasic), coumaphos, coumatetralyl, cumyluron, cyclosulfamuron, cyfluthrin, beta-cyfluthrin, cypermethrin, alpha-cypermethrin, beta-cypermethrin, theta-cypermethrin, cyprodinil, daimuron, 2,4-DB, deltamethrin, desmedipham, diafenthiuron, dichlobenil, dichlofluanid, dichlorophen, diclocymet, diclomezine, dicloran, diclosulam, dicofol, diethofencarb, difenacoum, difenoconazole, difethialone, diflubenzuron, diflufenican, dimefuron, dimethametryn, dimethomorph, diniconazole, dinitramine, dinobuton, dinoterb, diphacinone, dithianon, dithiopyr, diuron, dodemorph, dodemorph acetate, emamectin benzoate, endosulfan, epoxiconazole, ergocalciferol, esfenvalerate, ethalfluralin, etbametsulfuron-methyl, ethofumesate, ethoxysulfuron, etobenzanid, etoxazole, famoxadone, fenamidone, fenarimol, fenazaquin, fenbuconazole, fenbutatin oxide, fenchlorazole-ethyl, fenclorim, fenhexamid, fenoxaprop-P-ethyl, fenoxycarb, fenpiclonil, fenpyroximate, fentin acetate, fentin hydroxide, fentrazamide, fenvalerate, fipronil, flamprop-M-isopropyl, flamprop-M-methyl, flocoumafen, fluazinam, fluazolate, fluazuron, flucycloxuron, fludioxonil, flufenoxuron, flumetralin, flumetsulam, flumiclorac-pentyl, fluoroglycofen-ethyl, fluoroimide, fluquinconazole, flurazole, flurenol-butyl, fluridone, flurochloridone, fluroxypyr-meptyl, flurtamone, flusilazole, flusulfamide, fluthiacet-methyl, flutolanil, folpet, fomesafen, halofenozide, halosulfuron-methyl, haloxyfop, haloxyfop-etotyl, gamma-HCH, heptachlor, hexaconazole, hexaflumuron, hexythiazox, hydramethylnon, cyazofamid, imazosulfuron, imibenconazole, iminoctadine tris(albesilate), inabenfide, indanofan, indoxacarb, ioxynil, ipconazole, iprodione, iprovalicarb, isoxaben, isoxaflutole, kresoxim-methyl, lenacil, lufenuron, MCPA, mefenacet, mefenpyr-diethyl, mepanipyrim, mepronil, metconazole, methiocarb, methoxychlor, methoxyfenozide, metobenzuron, milbemectin, MK-616, 2-(1-naphthyl)acetamide, naproanilide, neburon, niclosamide, nitrothal-isopropyl, norflurazon, novaluron, nuarimol, oryzalin, oxabetrinil, oxadiargyl, oxadiazon, oxaziclomefone, oxolinic acid, oxpoconazole fumarate, oxyfluorfen, paclobutrazol, pencycuron, pendimethalin, pentanochlor, pentoxazone, permethrin, phenmedipham, N-phenylphthalamic acid, phosmet, phthalide, picobenzamid, picolinafen, picoxystrobin, pindone, polynactins, polyoxorim, primisulfuron-methyl, procymidone, prodiamine, prometryn, propaquizafop, propazine, propyzamide, prosulfuron, pyraflufen-ethyl, pyrazolynate, pyrazophos, pyrazosulfuron-ethyl, pyribenzoxim, pyributicarb, pyridaben, pyrimidifen, pyriminobac-methyl, quinclorac, quinoxyfen, quintozene, quizalofop-ethyl, quizalofop-P-ethyl, quizalofop-P-tefuryl, resmethrin, rimsulfuron, rotenone, siduron, silthiofam, simazine, spinosad, sulfluramid, sulfosulfuron, SZI-121, tebuconazole, tebufenozide, tebufenpyrad, tecloftalam, tecnazene, teflubenzuron, terbuthylazine, terbutryn, tetrachlorvinphos, tetradifon, tetramethrin, thenylchlor, thiabendazole, thiazopyr, thidiazuron, thifluzamide, thiodicarb, thiram, TI-35, tolclofos-methyl, tolylfluanid, tralkoxydim, tralomethrin, triadimenol, triasulfuron, triazoxide, tribenuron-methyl, trietazine, trifloxystrobin, triflumuron, triflusulfuron-methyl, triforine, triticonazole, uniconazole, uniconazole-P, vinclozolin, vitamin D3, warfarin, ziram, zoxamide, sulfaquinoxaline, aldrin, anilazine, barban, benodanil, benquinox, benzoylprop, benzoylprop-ethyl, binapacryl, bromofenoxim, bromophos, buturon, calcium cyanamide, camphechlor, chlobenthiazone, chlomethoxyfen, chlorbenside, chlorfenprop; chlorfenprop-methyl, chlornitrofen, chloromethiuron, chloroneb, chloropropylate, chloroxuron, chlorphoxim, climbazole, coumachlor, cyanofenphos, dialifos, dichione, diclobutrazol, dieldrin, dienochlor, difenoxuron, dioxabenzofos, dipropetryn, drazoxolon, fenitropan, fenoxaprop-ethyl; fenoxaprop, fenthiaprop; fenthiaprop-ethyl, flamprop-methyl; flamprop-isopropyl; flamprop, flubenzimine, fluenetil, flumipropyn, fluorodifen, fluotrimazole, flupoxam, forchlorfenuron, furconazole-cis, halacrinate, isomethiozin, isoxapyrifop, iodfenphos, leptophos, medinoterb acetate; medinoterb, methazole, methfuroxam, methoxyphenone, monalide, myclozolin, naphthalene, nitralin, nitrofen, phenisopham, phenylmercury dimethyldithiocarbamate, quinonamid, SMY 1500, tetcyclacis, tetrasul, thidiazimin, trichlamide, 2,2,2-trichloro-1-(3,4-dichlorophenyl)ethyl acetate, trifenmorph, urbacid.

A further preferred active substance-containing mixture is characterized in that the active substance is selected from the series of agents for curing, alleviating or preventing diseases in humans or animals such as, for example, therapeutic agents for acidosis, analeptics/antihypoxemics, analgesics/antirheumatics, anthelminthics, antiallergics, antianemics, antiarrhythmics, antibiotics/antiinfectives, antidementia drugs, antidiabetics, antidotes, antiemetics/antivertigo drugs, antiepileptics, antihemorrhagics, antihypertensives, antihypoglycemics, antihypotensives, anticoagulants, antimycotics, antiparasitic agents, antiinflammatory drugs, antitussives/expectorants, arteriosclerosis drugs, bronchodilators/antiasthmatics, cholagogs and biliary therapeutic agents, cholinergics, corticoids, dermatologicals, diuretics, blood flow-stimulating agents, anticraving drugs/agents for the treatment of addictive disorders, enzyme inhibitors, preparations for enzyme deficiency and transport proteins, fibrinolytics, geriatric drugs, antigout drugs, gynecologicals, hepatic drugs, hypnotics/sedatives, immunomodulators, cardiac drugs, coronary agents, laxatives, lipid-lowering agents, local anesthetics/neurotherapeutic agents, gastrointestinal drugs, migraine remedies, muscle relaxants, ophthalmologicals, osteoporosis remedies/calcium metabolism regulators, otologicals, psychoactive drugs, rhinologicals/sinusitis remedies, roborants/tonics, thyroid therapeutic agents, sex hormones and their inhibitors, spasmolytics/anticholinergics, platelet aggregation inhibitors, tuberculosis drugs, stimulants, urologicals, vein therapeutic agents, vitamins, cytostatics, other antineoplastic agents and protectives. Examples which may be mentioned in this connection are boldine, quinolones, felodipine, flurbiprofen, ibuprofen, ketoprofen, macrolides, nicardipine, nifedipine, nimodipine, nisoldipine, nitrendipine, norfloxacin, ofloxacin, paclitaxel, sulfonamides and tetracyclines.

The invention also relates to the use of the active substance-containing mixtures of the invention or of the active substance-containing mixtures obtainable by the process for preparing active substance-containing suspensions in water or aqueous solvents as crop protection agents, for example as sprays or soil treatment agents, and for producing pharmaceutical preparations, for example in oral dosage form.

Description of the Process Variants:

The process is preferably carried out in accordance with the following principle:
solution E is mixed with solution F, and the product obtained in this way is substantially dried.
Solution E consists of a solvent 1, dissolved therein the active substance A) and, where appropriate, the dispersant C) and, where appropriate, the polymer B).
Solution F: a displacement agent (solvent 2), dissolved therein the polymer and, where appropriate, the dispersant.
   The Solutions Ordinarily Comprise:
Solution E: solvent 1, active substance A) and dispersant C)
Solution F: displacement agent 2, polymer B)
   The Following Solutions are Sometimes Advantageous:
Solution E: solvent 1, active substance A), polymer B) and dispersant C)
Solution F: displacement agent 2, polymer B)
   The Following Are Also Possible:
Solution E: solvent 1, active substance A)
Solution F: displacement agent 2, polymer B) and dispersant C)
Displacement agent 2 is preferably water but can also be any other liquid which is completely miscible with solvent 1 and in which the solubility of the active substance A) is poor. Poor solubility means in this connection a solubility of less than 1% by weight, preferably less than 0.1% by weight, particularly preferably less than 0.01% by weight.

Suitable solvents 1 are all solvents miscible with the displacement agent 2. Particularly suitable solvents are those in which active substance A) shows a solubility of greater than 1% by weight, preferably greater than 10% by weight.

The mixing takes place for example by feeding solution E and F uniformly and continuously into a mixing chamber. To produce a homogeneous fine-particle suspension, it is expedient to produce a vigorous turbulence for intensive mixing. It is immaterial in this connection whether the turbulence is produced by a pressure drop in a mixing nozzle, by stirring, by ultrasound or in another way.

To improve the mixing it is expedient for the viscosity of both solutions to be less than 100 mPas, preferably less than 50 mPas, particularly preferably less than 20 mPas. It is likewise advantageous if the difference in the viscosity of the two solutions is small. The viscosity of the solutions can be adapted where appropriate by appropriately dividing the polymers B) between the two solutions or diluting the solutions appropriately.

It is possible alternatively with the processes identified above also to obtain a formulation by precipitating an active substance from its salt which is present in aqueous solution. It is immaterial in this connection whether the active substance is an acid which is displaced from its salt by adding a stronger acid. The active substance can equally be a base which is displaced from its salt by adding a stronger base. Accordingly, the solvent is to be understood as the aqueous base/acid which corresponds to the active substance and which dissolves the active substance by salt formation. Correspondingly, the displacement agent is to be understood as the aqueous solution of the stronger acid/base which displaces the active substance from its salt. The other statements apply correspondingly. Examples of acids which can be used include HCl, H₂SO₄, HNO₃ or HF. Examples of bases which can be used include NaOH, KOH, Ba(OH)₂ or Ca(OH)₂.

Drying of the resulting suspension can take place in a manner known per se by, for example, freeze drying, spray granulation and, in particular, spray drying.

To improve handling of the resulting product, it is expedient in a particularly preferred process to add, before the drying, a carrier which unites the nanoparticulate active substance particles to macroscopic particles. The amount of carrier for this purpose is favorably 10-30% by weight of the finished formulation. The choice of a suitable carrier takes place in a manner known per se and may be for example a mixture of talc and a polyethylene glycol, an additional polymer B) such as, for example, modified starch, or high molecular weight sugars. However, it is also possible in a simple manner for the carrier to be an excess of one of the polymers employed for the stabilization. Addition of the carrier serving the purpose of improving handling can take place before the mixing in one or both of the solutions identified above, or be blended in after the mixing before the drying of the fine-particle suspension.

The active substance formulations in powder form of the invention consist of individual primary particles which are substantially composed of a homogeneous mixture of the active substance, of the dispersant and of the polymer. The particles are predominantly present in the amorphous state and have an average diameter in the nanometer range. Thus, the average particle diameter is generally between 20 and 2000 nm, preferably between 50 and 1000 nm.

The formulations of the invention are redispersible powders which consist of fine-particle active substance particles and are embedded where appropriate in a carrier.

The powder formulations of the invention are stable even on prolonged storage (e.g. 1 year). They can be converted by stirring in water into homogeneous suspensions with a primary particle size of less than 5 μm, or release the fine-particle active substance particles again on contact with body fluids after application.

The application rate of the powder formulations of the invention may vary within a relatively wide range. It depends on the active substances present in each case and on the content thereof in the formulations.

It is possible with the aid of the powder formulations of the invention to use active substances in a particularly advantageous manner. The contained active substances are readily bioavailable and display a biological activity which is substantially better than that of conventional formulations in which the active components are present in the crystalline state.

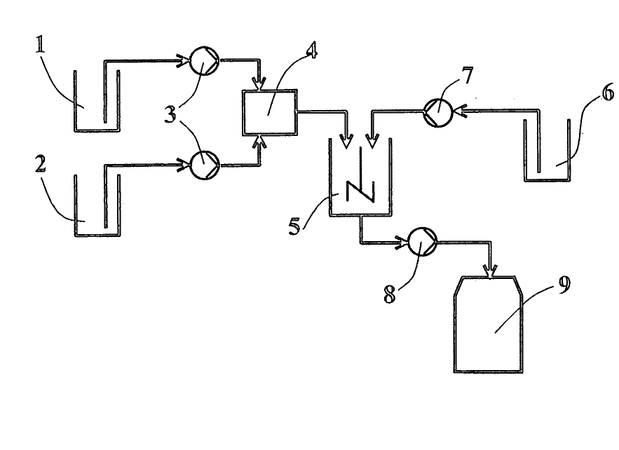

The invention is explained in more detail by way of example by means of FIG. 1 below.

FIG. 1 shows a diagram of an apparatus suitable for carrying out the process.

### EXAMPLES

Description of the Apparatus

The apparatus preferably used for carrying out the process of the invention is depicted diagrammatically in FIG. 1. The meanings in this figure are
1=receiver for solution E)
2=receiver for solution F)
3=pumps to raise the pressure
4=mixing chamber
5=collecting tank, expediently equipped with a stirrer
6=receiver for additives and carriers
7=delivery pump
8=delivery pump
9=dryer
   Process Procedure

Solution E) is introduced into receiver 1 and adjusted to the required temperature where appropriate to improve the solubility or reduce the viscosity. The temperature can be any temperature, preferably between 20° C. and the boiling point of the solvent, but can also be higher, for which purpose the appropriate pressure is adjusted in receiver 1.

Solution F) is introduced into tank 2 and adjusted to the desired temperature where appropriate to improve the solubility or reduce the viscosity. The temperature can be any temperature, preferably between 20° C. and the boiling point of the displacement agent, but can also be higher, for which purpose the appropriate pressure is adjusted in tank 2.

The pumps 3 for raising the pressure should operate with minimal pulsation, and gear pumps are favorable. Pumps associated with pulsation are also possible if the pulsation is reduced by an appropriate compensator. A pressure drop across the mixing chamber of 10-12 bar is normally sufficient for the mixing, but it is advantageous to increase the pressure drop across the mixing chamber to 30-50 bar for viscosities greater than 20 mPas. Greater pressures are also possible.

The collecting tank 5 may be operated discontinuously and continuously. In the discontinuous mode of operation, the collecting tank may be either empty or charged with the desired additives before the start of the experiment. The additives can also be fed from the receiver 6 with the aid of the delivery pump 7 uniformly together with the nanodisperse suspension from the mixing chamber 4 to the collecting tank 5.

The chosen residence time of the suspension in the collecting tank 5 should be as short as possible. A residence time of less than 30 min is beneficial, preferably less than 10 min. The nanodisperse suspension from the mixing chamber 4 can also be fed directly to the dryer 9 if no further additives are admixed.

The drying temperature depends on the boiling points of the solvent and of the displacement agent. The drying can be operated at atmospheric pressure or reduced pressure. A temperature below 80° C. is normally chosen, preferably below 50° C. The drying can also take place by freeze drying.

Analytical Methods

Analysis of the particle size distribution by laser diffraction, Malvern Mastersizer 2000, and photon correlation spectroscopy, Brookhaven Instruments BI-9000, cf. T. Allen, Particle size measurement, Vol. 1, 5th Ed., Kluwer Academic Publishers, Dordrecht, 1999.

Differential thermal analysis to determine the degree of crystallinity, Setaram C 80 II, Mettler, heatings between −100° C. and +250° C., heating rate 10 K/min

Preparation Examples

The following substances are used in the following examples:
N2-(1,1-Dimethyl-2-methylsulfonylethyl)-3-iodo-N1-{2-methyl-4-[1,2,2,2-tetrafluoro-1-(trifluoro-methyl)ethyl]phenyl}phthalamide
Alkyl polyglycoside Glucopon© 600 CS UP (CASR No. 110615-47-9, from Cognis) N-Methylpyrrolidone ppa
Polyvinylpyrrolidone K30 (CAS No. 9003-39-8, from FLUKA)
Polyvinyl alcohol Mowiol© 3-83 (from Clariant)
PLURAFAC® LF 132 (from BASF)
Fluoxastrobin: (5,6-dihydro-1,4,2-dioxazine-3-yl)(2-((6-(2-chlorophenoxy)-5-fluoro-4-pyrimidinyl)oxy)phenyl)methanone o-methyloxime
Acetone ppa
Soprophor® 3D-33: phosphoric acid mono/diester mixture of a tristyrylphenol ethoxylate, approx. 16 EO (from Rhodia)
Modified starch HI-CAP® 100 (from National Starch & Chemical)
Genapol© C 100: (from Clariant)
Prothioconazole: 2-[2-(1-chlorocyclopropyl)-3-(2-chlorophenyl)-2-hydroxypropyl]-1,2-dihydro-3H-1,2,4-triazole-3-thione
Sodium hydroxide solution, NaOH aqueous
Sulfuric acid, H₂SO₄ aqueous

### EXAMPLES

### Example 1

### Insecticide N2-(1,1-dimethyl-2-methylsulfonylethyl)-3-iodo-N1-{2-methyl-4-[1,2,2,2-tetrafluoro-1-(trifluoromethyl)ethyl]phenyl}phthalamide

Solution E: 12 g of N2-(1,1-dimethyl-2-methylsulfonylethyl)-3-iodo-N1-{2-methyl-4-[1,2,2,2-tetrafluoro-1-(trifluoromethyl)ethyl]phenyl}phthalamide, 12 g of alkyl polyglycoside Glucopon© 600 CS UP are dissolved in 54 g of N-methylpyrrolidone at 20° C.

Solution F: 12 g of polyvinylpyrrolidone K30, 12 g of polyvinyl alcohol Mowiol© 3-83 are dissolved in 198 g of deionized water under ambient conditions.

Solution E is fed at 10 kg/h and solution F at 32 kg/h into the mixing chamber and mixed turbulently to set up a mixing ratio of 1/3.2.

The suspension is collected without further additives in a glass beaker.

The resulting suspension has an average diameter of the suspended primary particles of 0.94 μm (measurement by laser diffraction).

The suspension is added dropwise to liquid nitrogen, and the resulting solid is freeze-dried.

The resulting product is amorphous according to DSC measurements.

### Example 2

The procedure was as in Example 1 but with an additive to promote penetration:

Plasticizer PLURAFAC® LF 132

Solution E: 12 g of N2-(1,1-dimethyl-2-methylsulfonylethyl)-3-iodo-N1-{2-methyl-4-[1,2,2,2-tetrafluoro-1-(trifluoromethyl)ethyl]phenyl}phthalamide, 12 g of alkyl polyglycoside Glucopon© 600 CS UP are dissolved in 54 g of N-methylpyrrolidone under ambient conditions.

Solution F: 12 g of polyvinylpyrrolidone K30, 12 g of polyvinyl alcohol Mowiol© 3-83 are dissolved in 198 g of deionized water under ambient conditions.

Solution E is fed at 12 kg/h and solution F at 35 kg/h into the mixing chamber to set up a mixing ratio of 1/2.92.

The suspension is collected in a glass beaker and mixed with 24 g of plasticizer PLURAFAC® LF 132 and 24 g of polyvinyl alcohol Mowiol© 3-83.

The suspension is quenched in liquid nitrogen and freeze dried.

The resulting product is amorphous according to DSC.

To examine the stability, a sample is stored at 54° C. for 2 weeks, and the sample remains in the amorphous state according to DSC measurement.

A check after storage under ambient conditions for 34 weeks revealed that the sample is still stably in the amorphous state.

### Example 3

The procedure was as in Example 1, but with a different mixing ratio.

Solution E: 12 g of N2-(1,1-dimethyl-2-methylsulfonylethyl)-3-iodo-N1-{2-methyl-4-[1,2,2,2-tetrafluoro-1-(trifluoromethyl)ethyl]phenyl}phthalamide, 12 g of alkyl polyglycoside Glucopon© 600 CS are dissolved in 54 g of N-methylpyrrolidone under ambient conditions.

Solution F: 12 g of polyvinylpyrrolidone K30, 12 g of polyvinyl alcohol Mowiol© 3-83 are dissolved in 330 g of deionized water under ambient conditions.

Solution E is fed at 7 kg/h and solution F at 40 kg/h into the mixing chamber to set up a mixing ratio of 0.175/1.

The suspension is collected without further additives in a glass beaker.

The resulting suspension has an average diameter of 0.95 μm (laser diffraction)

The resulting suspension is quenched in liquid nitrogen and freeze dried.

An amorphous product is obtained.

To examine the stability, a sample is stored at 54° C. for 2 weeks, and the sample remains in the amorphous state according to DSC.

### Example 4

The procedure was as in Example 1, but a different active substance was employed:

Fluoxastrobin, melting point 101° C.

Solution E: 60 g of fluoxastrobin, 40 g of acetone, 45 g of Soprophor® 3D-33

Solution F: 45 g of polyvinylpyrrolidone K30, 45 g of HI-CAP© 100, 345 g of deionized water

Solution E is fed at 5.7 kg/h and solution F at 15.3 kg/h into the mixing chamber to set up a mixing ratio of 1/2.68.

The suspension is collected without further additives in a glass beaker.

The resulting suspension is quenched in liquid nitrogen and freeze dried.

The resulting product is amorphous according to DSC measurement.

### Example 5

The procedure was as in Example 4 but based on a different mixing ratio and different additives.

Solution E: 49.5 g of fluoxastrobin, 100.5 g of acetone, 37.1 g of Soprophor® 3D-33

Solution F: 37.1 g of polyvinylpyrrolidone K30, 123.8 g of Mowiol© 3-83, 774.6 g of deionized water

Solution E is fed at 5.7 kg/h and solution B at 27 kg/h into the mixing chamber to set up a mixing ratio of 1/4.74.

The resulting suspension has an average diameter of 0.30 μm (LCS)

The suspension is collected in a glass beaker and mixed with a solution of 198 g of plasticizer Genapol© C 100 and 594 g of water.

The suspension is quenched in liquid nitrogen and freeze dried.

An amorphous product is obtained.

### Example 6

A different active substance was employed in this case:

Prothioconazole, melting point 140° C. In addition, a different process was used for precipitation from the salt of the active substance.

Solution E: 25 g of prothioconazole, 44 g of 10% by weight sodium hydroxide solution, 12.5 g of Soprophor® 3D-33, diluted to 250 ml with deionized water.

Solution F: 49 g of 10% by weight sulfuric acid, 25 g of polyvinylpyrrolidone K30, 25 g of Mowiol© 3-83, diluted to 250 ml with deionized water.

Solution E is fed at 5 l/h and solution B at 5 l/h into the mixing chamber and turbulently mixed to set up a volumetric mixing ratio of 1/1.

This suspension is collected without further additives in a glass beaker.

The resulting suspension has a pH=4.7 and an average diameter of 0.21 μm (LCS)

The resulting suspension is quenched in liquid nitrogen and freeze dried.

An amorphous product is obtained.

To examine the stability, a sample is stored at 54° C. for 2 weeks, and the sample remains in the amorphous state according to DSC.

### USE EXAMPLES

### Use Example 1

The insecticidal effect of the formulations from preparation examples 1-3 can be shown in the biological test of the xylem-systemic activity.

For this purpose, all the samples were adjusted to a uniform concentration ratio of the penetration aid PLURAFAC® LF 132 to active substance in proportions of 2/1: e.g.: 25.58 mg of preparation example 1 (16.3% active substance content) plus 8.34 mg of PLURAFAC® LF 132. The appropriate proportion of PLURAFAC® LF 132 is already present in preparation example 2. The mixtures were made up to 10 ml with water and stirred so that all the samples comprise a final concentration of 417 mg/l active substance and 834 mg/l PLURAFAC® LF 132.

Live maize plants (2-3 leaves) were transferred from soil into 20 ml test vessels. An application zone was demarcated with a fat barrier in the lower third of the second leaf. 30 ml of a 417 ppm active substance spray solution were applied with a pipette, which corresponds to an approximate application rate of 250 g of active substance/ha. After 48 h, the part of the leaf above the application zone was cut off and divided into two parts, one proximal and one distal. These parts of the leaf were placed together with 3 L2 larvae of Spodoptera frugiperda in Petri dishes (filled with 4 ml of 1% agar). Feeding damage and mortality were assessed after three and five days. After three days, the larvae were fed with untreated maize leaves.

The biological activity of the tested formulations was very good.

### Use Example 2

Penetration test on preparation examples 4 and 5

This test measured the penetration of active substance through enzymatically isolated cuticles of apple tree leaves.

The leaves used were cut in the fully developed state off apple trees of the Golden Delicious variety. Isolation of the cuticles took place as follows
firstly leaf disks marked on the underside with dye and cut out were filled by vacuum infiltration with a pectinase solution (0.2 to 2% strength) buffered to a pH of between 3 and 4,
then sodium azide was added, and
the leaf disks treated in this way were left to stand until the original leaf structure had broken down and the non-cellular cuticle had detached.

Thereafter, only the cuticles which were from the upper sides of the leaf and were free of stomata and hairs were used further. They were washed several times alternately with water and a buffer solution of pH 7. The resulting clean cuticles were finally applied to small Teflon plates and smoothed and dried with a gentle stream of air.

In the next step, the cuticle membranes obtained in this way were placed for membrane transport investigations in diffusion cells (=transport chambers) made of stainless steel. For this purpose, the cuticles were placed centrally, using tweezers, on the silicone grease-smeared edges of the diffusion cells and sealed with a likewise greased ring. The arrangement was chosen thus in order that the morphological outer side of the cuticles faced upwards, i.e. towards the air, while the original inside faced the interior of the diffusion cell. The diffusion cells were filled with water or with a mixture of water and solvent.

To determine the penetration, 10 μl of a spray liquor of the composition specified below were applied to the outside of each cuticle.

Spray Liquor A (Invention)

Powder formulation of preparation example 5 in 1 liter of water.

Active substance content 1000 ppm

Spray Liquor B (Known)

Conventional suspension concentrate of the fungicidal active substance indicated in example 3 in 1 liter of water.
Active substance content 1000 ppm
   CIPAC water was used in each of the spray liquors.

After application of the spray liquors, the water was allowed to evaporate in each case, then the chambers were each turned over and placed in thermostated troughs, with a saturated aqueous calcium nitrate 4-hydrate solution being present underneath the outside of each cuticle. The penetration which started therefore took place at a relative humidity of 56% and a set temperature of 25° C. Samples were taken with a syringe at regular intervals and investigated by HPLC for the content of penetrated active substance.

The results of the experiment are evident from the table below. These stated numbers are averages of 8 measurements.

TABLE A

Active substance penetration
in % after exposure time

| | | | | |
|---|---|---|---|---|
| A | 3.85 h | 10.38 h | 16.87 h | 46.07 h |
| | 3.0% | 5.4% | 6.8% | 10.1% |
| B | 23.53 h | 46 h | 70.21 h | |
| | 0.63% | 0.72% | 1.16% | |

1. A process for preparing amorphous active substance formulations comprising the steps of
a) dissolving an active substance A) in a solvent 1, optionally together with a dispersing aid C) to form a solution E).
b) providing a liquid displacement agent 2in which the solubility of the active substance A) is less than 1% by weight and which is miscible with solvent 1 and which effects precipitation of the active substance A), as solution F.
c) adding a predominantly amorphous polymer B) which is readily soluble in water, to the solution from step a) and/or to solution F) from step b).
d) mixing solvent streams of solutions E) and F), optionally in a mixing nozzle, with the two streams being fed continuously and uniformly to the mixing zone of the mixing nozzle, optionally forming a turbulent flow in the mixing zone.
e) removing the solvents from the mixture by freeze drying, spray drying or spray granulation.

2. The process as claimed in claim 1, wherein the mixing in step d) and optionally the formation of a turbulent flow is effected in a mixing nozzle by a pressure gradient across the mixing nozzle, or by stirring or by ultrasound treatment of the mixed streams.

3. The process as claimed in claim 1, wherein the viscosity of solutions E) and F) is kept below 100 mPas.

4. The process of claim 1, wherein the displacement agent 2 is water or an aqueous solution of an acid, of a base or of a salt.

5. The process of claim 1, wherein the solvent 1 is a low molecular weight organic solvent or an aqueous solution of a base or of an acid.

6. The process of claim 1, wherein the drying step e) is preceded by addition to the suspension of from 10 to 30% by weight of a carrier selected from the group consisting of talc, polyethylene glycol, modified starch and high molecular weight sugar, and optionally further polymer B), in each case said amount being based on the total weight of the formulation.

7. A predominantly amorphous active substance formulation, comprising
0.5 to 50% by weight of an active substance A) which is crystalline at 50° C.,
50 to 90% by weight of a polymer B), selected from the group consisting of dextrans, dextrins, gum Arabic, polyvinyl alcohol, polyvinylpyrrolidone, polyethylene glycol, polyaspartic acid and alginates
and, based on the proportion of active substance A), 0.1 to 5 parts of a dispersing agent C), selected from the group consisting of nonionic, anionic, cationic and zwitterionic surface-active compounds,
wherein the formulation comprises homogeneous primary particles of a mixture of the substances A), B), C) having an average particle diameter of <5 μm and wherein more than 50% of the active substance A) therein is present in the amorphous state.

8. The predominantly amorphous active substance formulation of claim 7, wherein the dispersing aid C) is selected from the group consisting of products of the reaction of fatty acids, fatty acid esters, fatty alcohols, fatty amines, alkylphenols or alkylarylphenols with ethylene oxide and/or propylene oxide, and their sulfuric esters, phosphoric acid, monoesters and phosphoric diesters, products of the reaction of ethylene oxide with propylene oxide alkylsulfonates, alkyl sulfates, aryl sulfates, alkylaryl sulfates, alkyl ether sulfates, alkylaryl ether sulfates, tetraalkylammonium halides, trialkylarylammonium halides, alkylaryl ethoxylate, sorbitan ethoxylates and alkylamine sulfonates, individually or in any mixture.

9. The predominantly amorphous active substance formulation of claim 7, wherein the active substance is selected from the group consisting of crop protection agents, bird repellents, plant nutrients and soil conditioners,
and agents for curing, alleviating or preventing diseases in humans or animals.

10. Crop protection agents in the form of active substance-containing suspensions in water or aqueous solvents and pharmaceutical preparations in oral dosage form, comprising the predominantly amorphous active substance formulation of claim 7.

11. The process of claim 1, wherein said polymer B) is selected from the group consisting of dextrans, dextrins, gum Arabic, polyvinyl alcohol, polyvinylpyrrolidone, polyethylene glycol, polyaspartic acid and alginates,

12. The process of claim 5, wherein said organic solvent is selected from the group consisting of short-chain alcohols having 1 to 10 carbon atoms, short-chain glycols ketones having 3 to 10 carbon atoms, carboxylic acids, ethers, esters, heterocyclic amines, formamides, n-methylpyrrolidone, and dimethyl sulfoxide.

13. The process of claim 12, wherein said alcohols are selected from the group consisting of methanol, ethanol and 2-propanol, said glycols are selected from the group consisting of ethylene glycol and 1,2-propylene glycol, said ketones are selected from the group consisting of acetone and 2-butanone, said carboxylic acids are acetic acid, said ethers are selected from the group consisting of diethyl ether, tetrahydrofuran and methyl tert-butyl ether, said esters are selected from the group consisting of methyl acetate, ethyl actetate and methyl formate, said heterocyclic amines are pyridines and said formamides are dimethylformamide.

14. The predominantly amorphous active substance formulation of claim 9, wherein said crop protection agents are selected from the group consisting of herbicides, fungicides, insecticides, acaricides and nematicides, said soil conditioners are selected from the group consisting of bistrifluron, foramsulfuron, mesosulfuron-methyl, pyraclostrobin, pyriftalid, abamectin, AC 94,377, acequinocyl, acibenzolar-S-methyl, aclonifen, acrinathrin, AKH-7088, amidosulfuron, amitraz, anilofos, anthraquinone, atrazine, azafenidin, azinphos-methyl, azocyclotin, azoxystrobin, beflubutamid, benalaxyl, benazolin-ethyl, benfluralin, benomyl, benoxacor, bensulfuron-methyl, bensultap, benzobicyclon, benzofenap, benzoximate, bifenazate, bifenox, bifenthrin, bitertanol, brodifacoum, bromadiolone, bromethalin, bromobutide, bromopropylate, bromuconazole, bupirimate, buprofezin, butafenacil, butralin, butroxydim, cafenstrole, captafol, captan, carbendazim, carpropamid, chinomethionat, chlorbromuron, chlordane, chlorfluazuron, chlorflurenol-methyl, chlorimuron-ethyl, chlorothalonil, chlorthal-dimethyl, chlozolinate, chromafenozide, cinidon-ethyl, clodinafop-propargyl, clofentezine, clomeprop, cloquintocet-mexyl, cloransulam-methyl, copper oxychloride, copper sulfate (tribasic), coumaphos, coumatetralyl, cumyluron, cyclosulfamuron, cyfluthrin, beta-cyfluthrin, cypermethrin, alpha-cypermethrin, beta-cypermethrin, theta-cypermethrin, cyprodinil, daimuron, 2,4-DB, deltamethrin, desmedipham, diafenthiuron, dichlobenil, dichlofluanid, dichlorophen, diclocymet, diclomezine, dicloran, diclosulam, dicofol, diethofencarb, difenacoum, difenoconazole, difethialone, diflubenzuron, diflufenican, dimefuron, dimethametryn, dimethomorph, diniconazole, dinitramine, dinobuton, dinoterb, diphacinone, dithianon, dithiopyr, diuron, dodemorph, dodemorph acetate, emamectin benzoate, endosulfan, epoxiconazole, ergocalciferol, esfenvalerate, ethalfluralin, ethametsulfuron-methyl, ethofumesate, ethoxysulfuron, etobenzanid, etoxazole, famoxadone, fenamidone, fenarimol, fenazaquin, fenbuconazole, fenbutatin oxide, fenchlorazole-ethyl, fenclorim, fenhexamid, fenoxaprop-P-ethyl, fenoxycarb, fenpiclonil, fenpyroximate, fentin acetate, fentin hydroxide, fentrazamide, fenvalerate, fipronil, flamprop-M-isopropyl, flamprop-M-methyl, flocoumafen, fluazinam, fluazolate, fluazuron, flucycloxuron, fludioxonil, flufenoxuron, flumetralin, flumetsulam, flumiclorac-pentyl, fluoroglycofen-ethyl, fluoroimide, fluquinconazole, flurazole, flurenol-butyl, fluridone, flurochloridone, fluroxypyr-meptyl, flurtamone, flusilazole, flusulfamide, fluthiacet-methyl, flutolanil, folpet, fomesafen, halofenozide, halosulfuron-methyl, haloxyfop, haloxyfop-etotyl, gamma-HCH, heptachlor, hexaconazole, hexaflumuron, hexythiazox, hydramethyinon, cyazofamid, imazosulfuron, imibenconazole, iminoctadine tris(albesilate), inabenfide, indanofan, indoxacarb, ioxynil, ipconazole, iprodione, iprovalicarb, isoxaben, isoxaflutole, kresoxim-methyl, lenacil, lufenuron, MCPA, mefenacet, mefenpyr-diethyl, mepanipyrim, mepronil, metconazole, methiocarb, methoxychlor, methoxyfenozide, metobenzuron, milbemectin, MK-616, 2-(1-naphthyl)acetamide, naproanilide, neburon, niclosamide, nitrothal-isopropyl, norflurazon, novaluron, nuarimol, oryzalin, oxabetrinil, oxadiargyl, oxadiazon, oxaziclomefone, ooxolinic acid, oxpoconazole fumarate, oxyfluorfen, paclobutrazol, pencycuron, pendimethalin, pentanochlor, pentoxazone, permethrin, phenmedipham, N-phenylphthalamic acid, phosmet, phthalide, picobenzamid, picolinafen, picoxystrobin, pindone, polynactins, polyoxorim, primisulfuron-methyl, procymidone, prodiamine, prometryn, propaquizafop, propazine, propyzamide, prosulfuron, pyraflufen-ethyl, pyrazolynate, pyrazophos, pyrazosulfuron-ethyl, pyribenzoxim, pyributicarb, pyridaben, pyrimidifen, pyriminobac-methyl, quinclorac, quinoxyfen, quintozene, quizalofop-ethyl, quizalofop-P-ethyl, quizalofop-P-tefuryl, resmethrin, rimsulfuron, rotenone, siduron, silthiofam, simazine, spinosad, sulfluramid, sulfosulfuron, SZI-121, tebuconazole, tebufenozide, tebufenpyrad, tecloftalam, tecnazene, teflubenzuron, terbuthylazine, terbutryn, tetrachlorvinphos, tetradifon, tetramethrin, thenylchior, thiabendazole, thiazopyr, thidiazuron, thifluzamide, thiodicarb, thiram, TI-35, tolclofos-methyl, tolylfluanid, tralkoxydim, tralomethrin, triadimenol, triasulfuron, triazoxide, tribenuron-methyl, trietazine, trifloxystrobin, triflumuron, triflusulfuron-methyl, triforine, triticonazole, uniconazole, uniconazole-P, vinclozolin, vitamin D3, warfarin, ziram, zoxamide, sulfaquinoxaline, aldrin, anilazine, barban, benodanil, benquinox, benzoylprop; benzoylprop-ethyl, binapacryl, bromofenoxim, bromophos, buturon, calcium cyanamide, camphechlor, chlobenthiazone, chlomethoxyfen, chlorbenside, chlorfenprop; chlorfenprop-methyl, chlornitrofen, chloromethiuron, chloroneb, chloropropylate, chloroxuron, chlorphoxim, climbazole, coumachlor, cyanofenphos, dialifos, dichlone, diclobutrazol, dieldrin, dienochlor, difenoxuron, dioxabenzofos, dipropetryn, drazoxolon, fenitropan, fenoxaprop-ethyl; fenoxaprop, fenthiaprop; fenthiaprop-ethyl, flamprop-methyl; flamprop-isopropyl; flamprop, flubenzimine, fluenetil, flumipropyn, fluorodifen, fluotrimazole, flupoxam, forchlorfenuron, furconazole-cis, halacrinate, isomethiozin, isoxapyrifop, iodfenphos, leptophos, medinoterb acetate; medinoterb, methazole, methfuroxam, methoxyphenone, monalide, myclozolin, naphthalene, nitralin, nitrofen, phenisopham, phenylmercury dimethyldithiocarbamate, quinonamid, SMY 1500, tetcyclacis, tetrasul, thidiazimin, trichlamide, 2,2,2-trichloro-1-(3,4-dichlorophenyl)ethyl acetate, trifenmorph, urbacid, said agents for curing, alleviating or preventing diseases in humans or animals are selected from the group consisting of therapeutic agents for acidosis, analeptics/antihypoxemics, analgesics/antirheumatics, anthelminthics, antiallergics, antianemics, antiarrhythmics, antibiotics/antiinfectives, antidementia drugs, antidiabetics, antidotes, antiemetics/antivertigo drugs, antiepileptics, antihemorrhagics, antihypertensives, antihypoglycemics, antihypotensives, anticoagulants, antimycotics, antiparasitic agents, antiinflammatory drugs, antitussives/expectorants, arteriosclerosis drugs, bronchodilators/antiasthmatics, cholagogs and biliary therapeutic agents, cholinergics, corticoids, dermatologicals, diuretics, blood flow-stimulating agents, anticraving drugs/agents for the treatment of addictive disorders, enzyme inhibitors, preparations for enzyme deficiency and transport proteins, fibrinolytics, geriatric drugs, antigout drugs, gynecologicals, hepatic drugs, hypnotics/sedatives, immunomodulators, cardiac drugs, coronary agents, laxatives, lipid-lowering agents, local anesthetics/neurotherapeutic agents, gastrointestinal drugs, migraine remedies, muscle relaxants, ophthalmologicals, osteoporosis remedies/calcium metabolism regulators, otologicals, psychoactive drugs, rhinologicals/sinusitis remedies, roborants/tonics, thyroid therapeutic agents, sex hormones and their inhibitors, spasmolytics/anticholinergics, platelet aggregation inhibitors, tuberculosis drugs, stimulants, urologicals, vein therapeutic agents, vitamins, cytostatics, other antineoplastic agents and protectives.

15. The predominantly amorphous active substance formulation of claim 14, wherein said agents for curing, alleviating or preventing diseases in humans or animals are selected from the group consisting of boldine, quinolones, felodipine, flurbiprofen, ibuprofen, ketoprofen, macrolides, nicardipine, nifedipine, nimodipine, nisoldipine, nitrendipine, norfloxacin, ofloxacin, paclitaxel, sulfonamides and tetracyclines.
